(19) United States
(12) Patent Application Publication
(10) Pub. No.: US 2012/0078219 A1
(43) Pub. Date: Mar. 29, 2012

(54) Syringe Assembly

(75) Inventor: Colin Preis, Hopkinton, MA (US)

(73) Assignee: Tyco Healthcare Group LP, Mansfield, MA (US)

(21) Appl. No.: 12/893,532

(22) Filed: Sep. 29, 2010

(51) Int. Cl. A61M 5/315

(52) U.S. Cl. 604/500; 604/218

(57) ABSTRACT

A syringe assembly includes a syringe body having an internal fluid reservoir and a fluid contained within the fluid reservoir and defining a fluid outlet adjacent the distal end thereof, a plunger at least partially disposed within the syringe body and having a plunger rod and a distal plunger head and being adapted for longitudinal movement from an initial retracted position to an actuated advanced position and a dispensing element disposed within the internal fluid reservoir of the syringe body and distal of the plunger head. The dispensing element is dimensioned and arranged to cooperate with the plunger head to permit engagement and advancing movement of the dispensing element during corresponding movement of the plunger head without effecting a coupling relationship with the plunger head whereby the plunger is movable toward the retracted position without causing corresponding movement of the dispensing element.

### BACKGROUND

1. Technical Field

The present disclosure generally relates to syringes and, in particular, relates to medical syringes such as disposable syringes and prefilled syringes, which may be discarded after a single administration.

2. Background

Single-use syringes are well known in the medical arts. A single-use syringe prevents reuse of the syringe to minimize exposure of patients to HIV, hepatitis and other blood-borne pathogens. A variety of different types of single-use syringes are known. These include syringes having frangible plunger disabling mechanisms and those having complex locking elements, e.g., precision metal stampings or the like. Those with complex locking mechanisms are expensive and require complicated manufacturing procedures. With regard to those syringes having disabling mechanisms, it has been difficult to engineer, design and manufacture such mechanisms to operate to eject fluid from a syringe while assuring disablement of the plunger during or after use.

Accordingly, a continuing need exists in the medical arts for a simple, reliable, robust single-use syringe.

### SUMMARY

In accordance with one embodiment of the present disclosure, a syringe assembly includes a syringe body having an internal fluid reservoir and a fluid contained within the fluid reservoir and defining a fluid outlet adjacent the distal end thereof, a plunger at least partially disposed within the syringe body and having a plunger rod and a distal plunger head and being adapted for longitudinal movement from an initial retracted position to an actuated advanced position, and a dispensing element disposed within the internal fluid reservoir of the syringe body and distal of the plunger head. The dispensing element is positioned to be engaged by the plunger head during movement of the plunger toward the advanced position thereof such that the dispensing element dispenses the fluid through the fluid outlet of the syringe body. The dispensing element is dimensioned and arranged to cooperate with the plunger head to permit engagement and advancing movement of the dispensing element during corresponding movement of the plunger head without effecting a coupling relationship with the plunger head whereby the plunger is movable toward the retracted position without causing corresponding movement of the dispensing element.

The dispensing element may include a plurality of longitudinally spaced external ribs whereby adjacent ribs define recesses therebetween. The ribs and recesses cooperate to reduce forces required to move the dispensing element within the syringe body. The dispensing element may define a substantially frusto-conical shaped distal face.

A conduit may be connected adjacent the distal end of the syringe body and may define a lumen in fluid communication with the internal fluid reservoir. The conduit may include a distal penetrating end.

In accordance with another embodiment, a syringe assembly includes a syringe body having an internal fluid reservoir and a fluid contained within the fluid reservoir and defining a fluid outlet adjacent the distal end thereof, a needled conduit connected adjacent the distal end of the syringe body and defining a lumen in fluid communication with the internal fluid reservoir, a plunger at least partially disposed within the syringe body and having a plunger rod and a distal plunger head and being adapted for longitudinal movement from an initial retracted position to an actuated advanced position, and a dispensing element disposed within the internal fluid reservoir of the syringe body distal of the plunger head. The dispensing element is positioned to be engaged by the plunger head during movement of the plunger toward the advanced position thereof such that the dispensing element dispenses the fluid through the fluid outlet of the syringe body and into the needled conduit. At least one of the dispensing element and the plunger head is devoid of irregularities to permit engagement and advancing movement of the dispensing element during corresponding movement of the plunger head without effecting a coupling relationship with the plunger head. In this manner, the plunger is movable toward the retracted position without causing corresponding movement of the dispensing element.

A surgical procedure is also provided. The surgical procedure includes the steps of:
providing a syringe assembly including a syringe body having an internal fluid reservoir, a plunger at least partially disposed within the syringe body and a dispensing element within the internal fluid reservoir distal of the plunger;
advancing the plunger relative to the syringe body from an initial position to an advanced position such that the plunger causes corresponding advancing movement of the dispensing element whereby the dispensing element expresses fluid from the internal fluid reservoir of the syringe body without; and
establishing a non coupling relationship between the plunger and the dispensing element such that subsequent to movement of the plunger to the advanced position, the plunger may be moved toward the initial position without causing corresponding withdrawal of the dispensing element thereby substantially preventing ingress of fluids within the internal fluid reservoir.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a side view of a syringe assembly illustrating the syringe body and the needle extending from the syringe body in accordance with the embodiments of the present disclosure;

FIG. 2 is an exploded view of the syringe assembly of FIG. 1;

FIG. 3 is a perspective view of the syringe body of the syringe assembly of FIG. 1;

FIG. 4 is a perspective view of the syringe body of FIG. 3 with a section of the syringe body removed, illustrating the dispensing element within the syringe body and the plunger;

FIG. 5 is a side view of the dispensing element;

FIGS. 6, 7, and 8 are cross-sectional views illustrating movement of the plunger between a retracted position and an advanced position;

FIG. 9 is an exploded view illustrating an alternate embodiment of the syringe assembly; and

FIG. 10 is an exploded perspective view of another alternative embodiment of the syringe assembly.

### DETAILED DESCRIPTION OF THE PREFERRED EMBODIMENT(S)

Embodiments of the presently disclosed plunger and syringe assembly will now be described in detail with reference to the drawings wherein like reference numerals designate identical or corresponding elements in each of the several views. In this description, the term “proximal” or “trailing” is generally used to indicate the relative nearness of a referenced item to a clinician using the assembly and the term “distal” or “heading” is used to indicate the remoteness of a referenced item to a clinician using the device.

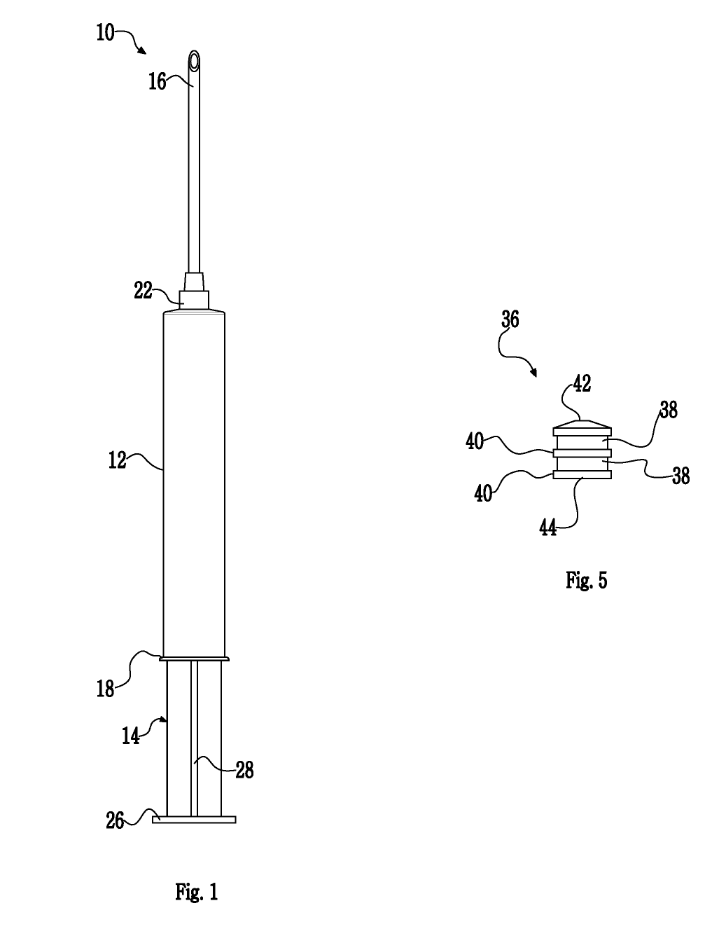

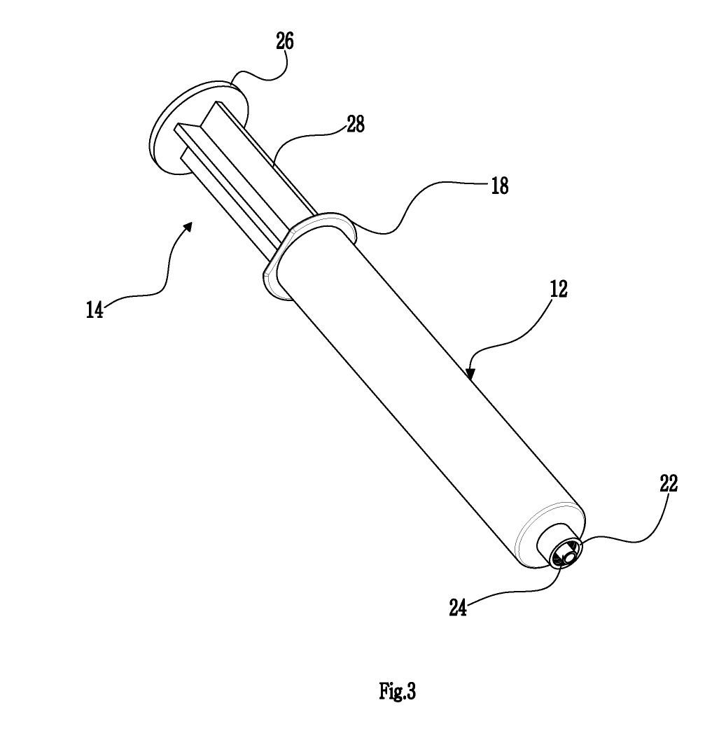

Referring now to the drawings wherein like components are designated by like reference numerals throughout the several views, FIGS. 1-4 illustrate a syringe assembly 10 in accordance with the principles of the present disclosure. In one embodiment, syringe assembly 10 may be a single-use assembly, i.e., disposable after a single application. In another embodiment, syringe assembly 10 may be a prefilled syringe assembly, in particular a prefilled sterilized syringe, for which the sterilized portion of the syringe assembly may not extend beyond a plunger tip into an unfilled portion of a syringe banal. In certain applications, syringe assembly 10 may contain a flushing solution, e.g., saline or the like. Alternatively, syringe assembly 10 may contain medicinals, including antibiotics, pain medication, therapeutic drugs, heparin or the like.

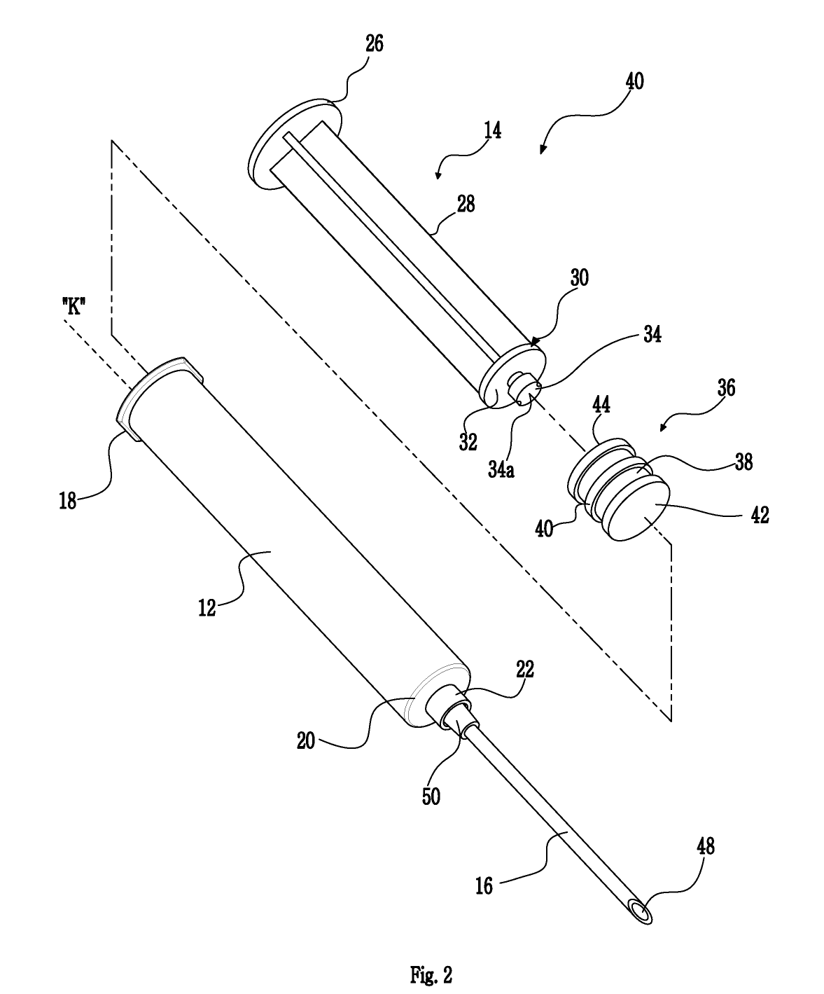

Syringe assembly 10 includes syringe body 12, plunger 14 at least partially received within syringe body 12 and needle 16 which is mounted to syringe body 12. As best depicted in FIGS. 2-4, syringe body 12 defines longitudinal axis “K” extending a length of syringe body 12. Syringe body 12 defines fluid chamber or internal reservoir 18 (see FIG. 4), which contains pre-filled medicinals “f”. Adjacent a proximal end of syringe body 12 is flange 18, which may be integrally formed with syringe body 12. Flange 18 may be relatively enlarged to enhance engagement by the clinician. Distal end 20 of syringe body 12 includes needle connector mechanism 22, which may, in one embodiment, be a luer connector or luer lock. Distal end 20 further defines fluid outlet 24 which is in fluid communication with internal reservoir 18.

Plunger 14 includes proximal flange 26 (see FIGS. 2 and 4), elongated plunger rod 28 extending from the proximal flange 26 and plunger head 30 (see FIGS. 2 and 4) at a distal end of plunger rod 28. Plunger flange 26 is enlarged to facilitate engagement by a clinician. Plunger rod 28 may have any cross-sectional dimension. In the embodiments depicted in FIGS. 1-4, plunger rod 28 has a “+” shaped cross-section. Plunger head 30 includes enlarged disc 32 having a cross-section or diameter, which generally approximates an internal diameter defined by an internal wall of syringe body 12. Plunger head 30 may further include protruding head segment 34 extending distally beyond enlarged disc 32. Head segment 34 may define any configuration, but is dimensioned to provide an atraumatic distal or leading profile. In one embodiment, head segment 34 defines a substantially planar distal face 34a. Head segment 34 may have other configurations including convex, concave or other shaped surface arrangements. It is further envisioned that plunger head 30 may be devoid of head segments 34, and consist entirely of plunger disc 32. Plunger head 30 may present a profile which is devoid of irregularities for reasons to be discussed hereinabove.

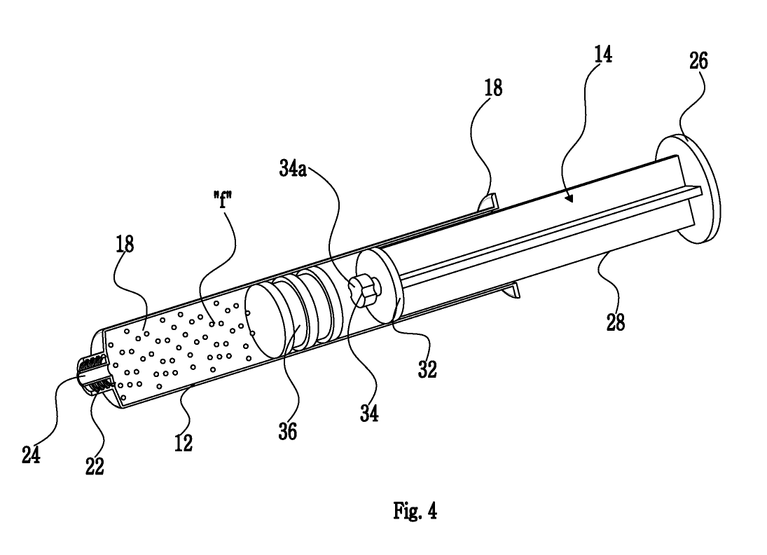

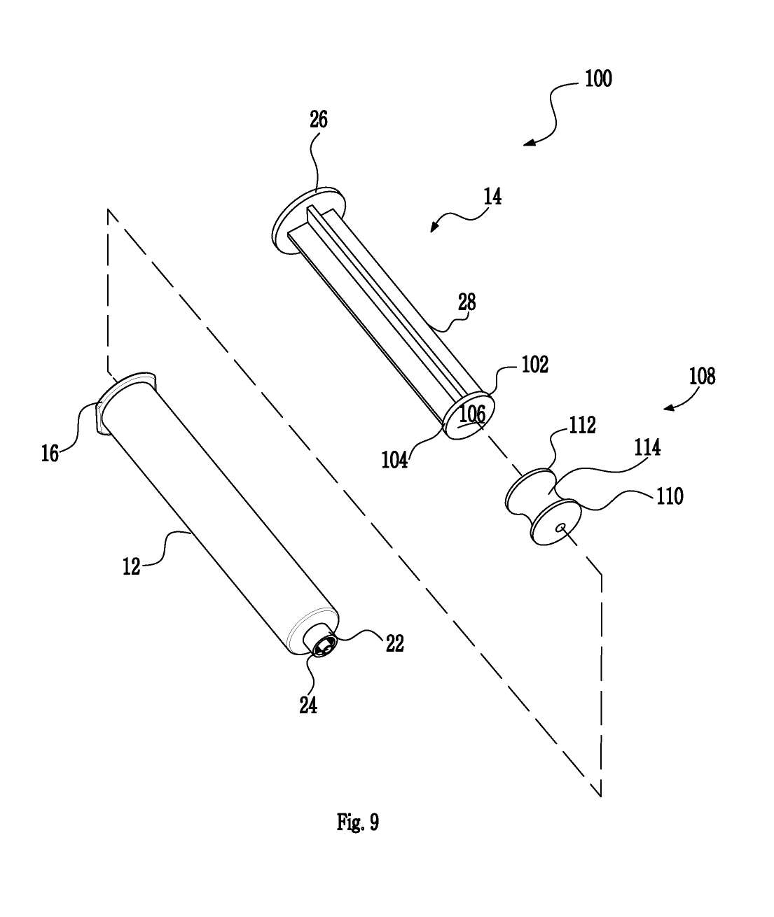

Referring to FIGS. 4 and 5, syringe assembly 10 further includes expressing or dispensing element 36 disposed within the syringe body and abuting reservoir 18 of syringe body 12. Dispensing element 36 may comprise an elastomeric material or the like. Dispensing element 36 defines, in one embodiment, a circular cross-sectional dimension, which generally approximates an inner dimension of syringe body 12. In general, dispensing element 36 is adapted to traverse or longitudinally move within syringe reservoir 18 upon corresponding longitudinal movement of plunger 14 to dispense fluids through fluid outlet 24 of syringe body 12. Dispensing element 36 may include annular recesses 38 (see FIG. 9) to reduce friction between dispensing element 36 and the internal wall of syringe body 12 to facilitate advancing movement. Annular recesses 38 are defined between adjacent ribs 40 of dispensing element 36. Dispensing element 36 may include a leading or distal conical face 42, which contacts the medicinal fluids within reservoir 18.

With continued reference to FIGS. 4 and 5, dispensing element 36 may include an atraumatic proximal or trailing face 44, which is contacted by head segment 34 of plunger head 30 during advancement of plunger 14. Trailing face 44 of dispensing element 36 may be planar or slightly arcuate, but is devoid of any recesses, depressions, undulations or other irregularities. In this manner, dispensing element 36 will not mechanically couple to, or with, plunger during any longitudinal movement of plunger 14. In particular, during advancing or distal movement of plunger 14, plunger head 30 including disc 32 and head segment 34, may contact trailing face 44 of dispensing element 36 to cause corresponding advancing movement of dispensing element 36 and subsequent dispensing of pre-filled solution “f”. However, trailing face 44 of dispensing element 36 will not couple with or become attached to plunger head 30. Accordingly, plunger 14 may be retracted without causing any retracting movement of dispensing element 36, i.e., dispensing element 36 will remain in an advanced position. More specifically, dispensing element 36 is dimensioned and arranged to cooperate with plunger head 30 to permit engagement and advancing movement of dispensing element 36 during corresponding movement of plunger head 30 without effecting a coupling relationship with plunger head 30, whereby plunger 14 is movable toward the retracted position without causing corresponding movement of dispensing element 36. This advantageously prevents use of syringe assembly 10 in any aspirating capacity, thereby preventing fluids to be drawn into syringe body 12.

Referring again to FIG. 1-2, needle or conduit 16 includes central lumen 46 which extends the length of the needle 16 and is in fluid communication with fluid outlet 24 of syringe body 12. Needle 16 includes penetrating end 48 which is adapted to pierce tissue for entry within the vessel. Needle 16 further includes connector 50 adjacent its proximal end. Connector 50 couples with connector 22 of syringe body 12 to connect needle 16 to the syringe body 12. Connector 50 may establish a releasable connection with connector 22 through a suer lock mechanism or the like, or may establish a more permanent connection with adhesives, cements, etc.

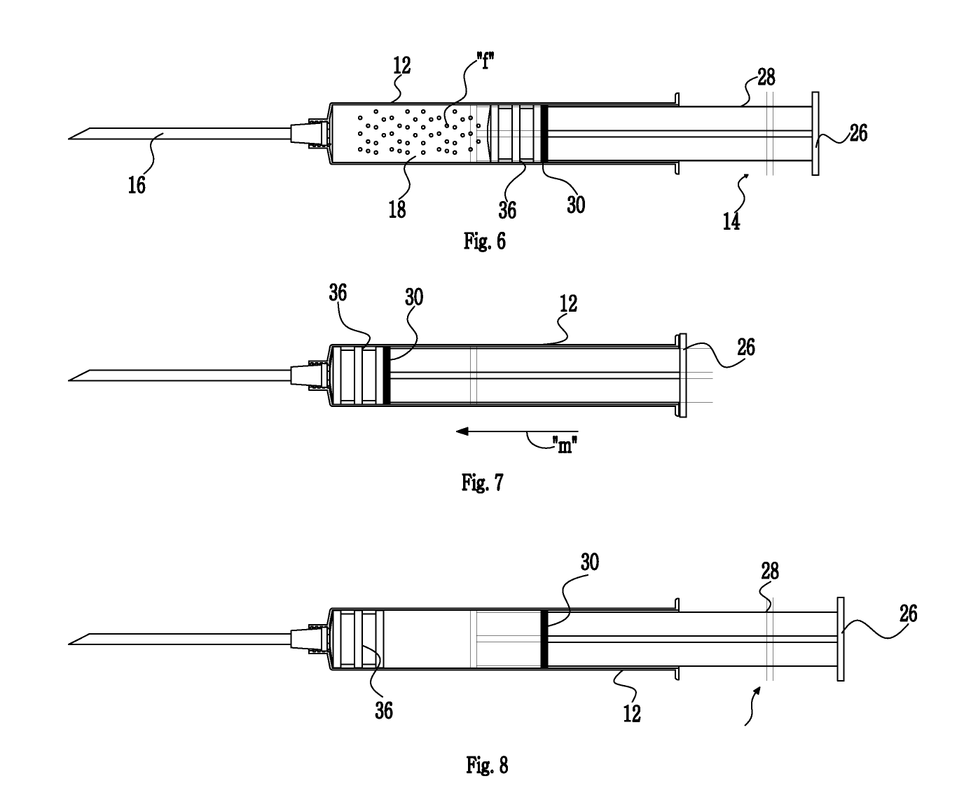

Referring to FIGS. 6-8, cross-sectional views of syringe assembly 10 illustrating movement of plunger 14 within syringe body 12 are illustrated. As illustrated in FIGS. 6-8, the embodiments of the present disclosure are constructed such that plunger 14 and dispensing element 36 cooperate to permit the dispensing element 36 to slidably move within reservoir 18 of syringe body 12. In a first position, depicted in FIG. 6, corresponding to a retracted position of plunger 14, dispensing element 36 is positioned adjacent plunger head 30 of plunger 14, i.e., the dispensing element 36 and plunger 14 are in a proximal most position with fluid “f” being within internal reservoir 18. In FIGS. 6-8, for illustrative purposes, plunger head 30 is shown without head segment 34.

During advancement of plunger 14 within syringe body 12 in the direction of directional arrows “m” as depicted in FIG. 7, plunger head 30 engages dispensing element 36 and causes corresponding advancement of the dispensing element 36. Such movement of the dispensing element 36 causes the fluid “f” within internal reservoir 18 to be dispensed through fluid outlet 24 and into needle 16. Plunger 14 is continually advanced to an actuated or fully advanced position, depicted in FIG. 7, to correspondingly move dispensing element 36 to express the entire volume of fluids “f” from internal reservoir 18.

As discussed hereinabove, during movement of the plunger 14, plunger head 30 and dispensing element 36 do not cooperate to form an integrally functioning assembly, i.e., plunger 14 and dispensing element 36 are separate and distinct, non-couplable elements and are devoid of irregularities which would otherwise connect the components, so that retraction of plunger 14 prior or subsequent to dispensing all or a portion of the fluid from the internal reservoir does not also retract the dispensing element . In one embodiment, once dispensing element 36 is driven to the distal most location, plunger 14 may be retracted toward its initial position without causing corresponding movement of the dispensing element 36. This substantially minimizes or removes the potential of syringe 10 to be used in an aspirating capacity, the benefits of which being hereinbelow discussed. Dispensing element 36 may be dimensioned to engage the internal wall of syringe body 12 in frictional relation therewith to be fixed in the distal position depicted in FIG. 8. In one embodiment, dispensing element 36 occupies the entire cross-sectional volume of internal reservoir 18 such that any potential vacuum, suction or aspirating forces created during a retracting movement of plunger 14 (e.g., subsequent to expressing the fluids “f”) in the opposite direction as also depicted in FIG. 8 is not communicated beyond the dispensing element 36 to fluid outlet 34.

Referring to FIG. 9, an exploded perspective view of an alternate embodiment of a syringe assembly 100 is illustrated. In accordance with this embodiment, plunger head 102 includes a plunger disc 104. Plunger disc 104 defines a leading or distal surface 106 which is devoid of irregularities. Dispensing element 108 defines leading (distal) and trailing (proximal) ends 110, 112 and central recessed segment 114. Leading and trailing ends 110, 112 define a cross-sectional dimension approximating the internal dimension of syringe body 12. Central recessed segment 114 reduces the level of force required to advance dispensing element 108 by minimizing the area of surface contact between the dispensing element 108 and the internal wall of syringe body 12. Trailing end 112 of dispensing element 108 may also be devoid of irregularities to ensure that there is no coupling relation between the dispensing element 108 and plunger head 102.

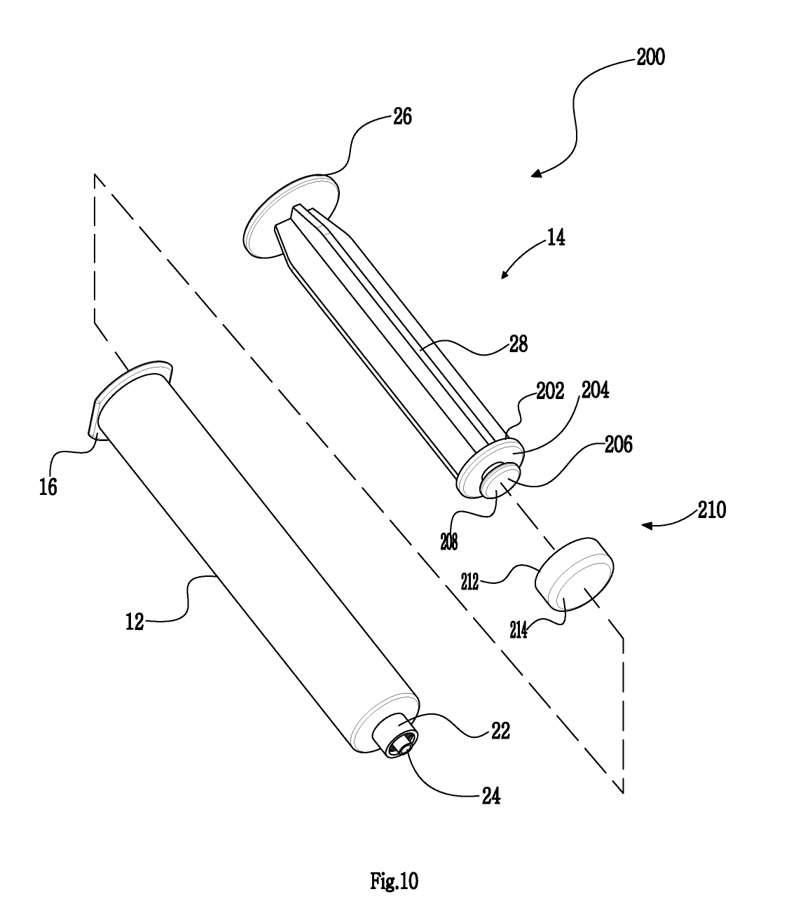

Referring to FIG. 10, an exploded perspective view of another embodiment of the syringe assembly is depicted. Syringe assembly 200 includes plunger head 202 having first and second disc elements 204, 206. Disc element 206 is the leading or distal disc and defines a smoothly planar end face 208. Dispensing element 210 is substantially cylindrical and defines a corresponding proximal end face 212 which is devoid of irregularities and may have a similar profile to distal end face 214. In this manner, dispensing element 210 will not couple with plunger head 202.

The advantages of the example embodiments of the present disclosure may include at least providing assurance of clinical observance of indications for use for prefilled flush syringes. The indications for use for prefilled flush syringes are limited to the flushing of I.V. access devices for the purpose of maintaining the patency of those devices. Clinicians sometimes use saline flush syringes to reconstitute lyophilized drugs. Clinicians perform such tasks by expressing the contents of the prefilled syringe into the vial containing the lyophilized drug. The clinicians aspirate the reconstituted drug back into the syringe for subsequent delivery to the patient. This is an off-label use and is not recommended.

The example embodiments of the present disclosure prevent the clinicians from using the same syringe for dispensing the solution into the drug vial and aspirating the reconstituted drug for delivery. By eliminating the apparent convenience of being able to use the syringe for all operations related to reconstituting and delivering a lyophilized drug, a clinician may be significantly discouraged from using the flush solution syringe for such application. The example embodiments of the present disclosure thus effectively prevent clinicians from reusing syringes for any purpose once the initial prefilled solution is expressed. In essence, the syringe has an auto-disable feature once the original solution has been expressed. Another advantage of some embodiments of the present invention is that the clinician is prevented from pulling back the plunger rod of a sterilized prefilled syringe into a non-sterile portion of the syringe barrel, thereby reducing or eliminating contamination during use and administration of the sterilized contents of the prefilled syringe.

Although the illustrative embodiments of the present disclosure have been described herein with reference to the accompanying drawings, it is to be understood that the disclosure is not limited to those precise embodiments, and that various other changes and modifications may be effected therein by one skilled in the art without departing from the scope or spirit of the disclosure.

What is claimed is:

1. A syringe assembly, which comprises:
a syringe body having an internal fluid reservoir and a fluid contained within the fluid reservoir, the syringe body defining a longitudinal axis, and proximal and distal ends, the syringe body defining a fluid outlet adjacent the distal end thereof;
a plunger at least partially disposed within the syringe body, the plunger including a plunger rod and a distal plunger head, the plunger adapted for longitudinal movement from an initial retracted position to an actuated advanced position; and
a dispensing element disposed within the syringe body and abutting the internal fluid reservoir of the syringe body and distal of the plunger head, the dispensing element positioned to be engaged by the plunger head during movement of the plunger toward the advanced position thereof such that the dispensing element dispenses the fluid through the fluid outlet of the syringe body, the dispensing element being dimensioned and arranged to cooperate with the plunger head to permit engagement and advancing movement of the dispensing element during corresponding movement of the plunger head without effecting a coupling relationship with the plunger head whereby the plunger is movable toward the retracted position without causing corresponding movement of the dispensing element.

2. The syringe assembly according to claim 1, wherein the dispensing element includes a plurality of longitudinally spaced external ribs, whereby adjacent ribs define recesses therebetween, the ribs and recesses cooperating to reduce forces required to move the dispensing element within the syringe body.

3. The syringe assembly according to claim 1, wherein the dispensing element defines a substantially frusto-conical shaped distal face.

4. The syringe assembly according to claim 1, including a conduit connected adjacent the distal end of the syringe body and defining a lumen in fluid communication with the internal fluid reservoir.

5. The syringe assembly according to claim 4, wherein the conduit includes a distal penetrating end.

6. The syringe assembly according to claim 1, wherein the plunger head defines a substantially planar distal surface portion and the dispensing element defines a substantially planar proximal surface portion, the substantially planar surface portions cooperating without effecting the coupling relationship.

7. The syringe assembly according to claim 1, wherein the plunger head is a disc member, the disc member having a protrusion depending therefrom, the protrusion cooperating with the dispensing element without effecting a coupling relationship.

8. A syringe assembly, which comprises:
a syringe body having an internal fluid reservoir and a fluid contained within the fluid reservoir, the syringe body defining a longitudinal axis, and proximal and distal ends, the syringe body defining a fluid outlet adjacent the distal end thereof;
a needled conduit connected adjacent the distal end of the syringe body and defining a lumen in fluid communication with the internal fluid reservoir.
a plunger at least partially disposed within the syringe body, the plunger including a plunger rod and a distal plunger head, the plunger adapted for longitudinal movement from an initial retracted position to an actuated advanced position; and
a dispensing element disposed within the syringe body and abutting the internal fluid reservoir of the syringe body and distal of the plunger head, the dispensing element positioned to be engaged by the plunger head during movement of the plunger toward the advanced position thereof such that the dispensing element dispenses the fluid through the fluid outlet of the syringe body and into the needled conduit, at least one of the dispensing element and the plunger head being devoid of irregularities to permit engagement and advancing movement of the dispensing element during corresponding movement of the plunger head without effecting a coupling relationship with the plunger head whereby the plunger is movable toward the retracted position without causing corresponding movement of the dispensing element.

9. A surgical procedure, comprising the steps of:
providing a syringe assembly including a syringe body having an internal fluid reservoir, a plunger at least partially disposed within the syringe body and a dispensing element within the internal fluid reservoir distal of the plunger;
advancing the plunger relative to the syringe body from an initial position to an advanced position such that the plunger causes corresponding advancing movement of the dispensing element whereby the dispensing element expresses fluid from the internal fluid reservoir of the syringe body without; and
establishing a non coupling relationship between the plunger and the dispensing element such that subsequent to movement of the plunger to the advanced position, the plunger may be moved toward the initial position without causing corresponding withdrawal of the dispensing element thereby substantially preventing ingress of fluids within the internal fluid reservoir.
